(19) United States
(12) Patent Application Publication
(10) Pub. No.: US 2011/0033464 A1
(43) Pub. Date: Feb. 10, 2011

(54) IMMUNOGLOBULIN FUSION PROTEIN FORMULATIONS

(75) Inventors: Anthony BARRY, Winchester, MA (US); Thomas Crowley, Wilmington, MA (US); Daniel Dixon, Arlington, MA (US); Jennifer Juneau, Sterling, MA (US); Ajay Kumar, North Andover, MA (US); Li Li, Sudbury, MA (US); Nicholas Luksha, Malden, MA (US); Michael Shamashkin, Somerville, MA (US); Erin Soley, Lowell, MA (US); Nicholas Warne, Andover, MA (US); Chandra Webb, Pelham, NH (US)

Correspondence Address: WYETH LLC/FINNEGAN HENDERSON, LLP, 901 NEW YORK AVENUE, NW, WASHINGTON, DC, 20001-4413, US

(73) Assignee: Wyeth

(21) Appl. No.: 12/910,146

(22) Filed: Oct. 22, 2010

Related U.S. Application Data
(63) Continuation of application No. 11/562,299, filed on Nov. 21, 2006.
(60) Provisional application No. 60/739,271, filed on Nov. 22, 2005.

(51) Int. Cl. A61K 39/395

(52) U.S. Cl. 424/134.1

(57) ABSTRACT

The present invention provides compositions of Ig fusion proteins, especially compositions including an Ig fusion protein, a bulking agent, a disaccharide, a surfactant, and a buffer. In one aspect, these compositions are stable under long-term storage or at least one freeze/thaw cycle. The invention also provides methods of preparation of the Ig fusion protein compositions. In one aspect, compositions of the invention are lyophilized. In a further aspect, the compositions are lyophilized by a process that includes an annealing step.

This application claims priority to U.S. Application No. 60/739,271, filed Nov. 22, 2005, which is hereby incorporated by reference.

### FIELD OF THE INVENTION

The present invention relates to the field of protein formulations. More specifically, the invention relates to pharmaceutical compositions comprising immunoglobulin (Ig) fusion proteins.

### BACKGROUND

Advances in biotechnology have made it possible to produce a wide variety of proteins for pharmaceutical applications. After production, protein pharmaceuticals must often be stored prior to their use. Due in part to the fact that proteins are generally larger and more complex than “traditional” pharmaceuticals, formulation and processing of protein pharmaceuticals that are suitable for storage can be particularly challenging. For reviews of protein pharmaceutical formulation and process design, see Carpenter et al., Pharmaceutical Research 14:969-975 (1997); Wang, International Journal of Pharmaceutics 203:1-60 (2000); and Tang and Pikal, Pharmaceutical Research 21:191-200 (2004).

Several factors can be considered in designing formulations and processes for protein pharmaceutical production. Of primary concern is the stability of the protein through any or all of the manufacture, shipping, and handling steps, which may include preparation of the composition, freezing, drying, storage, shipping, reconstitution, freeze/thaw cycles, and post-reconstitution storage by the end user. Other potential considerations include ease and economy of manufacture, handling, and distribution; composition of the final product for patient administration; and ease of use by the end user, including solubility of the lyophilized formulation upon reconstitution.

Liquid formulations may satisfy certain objectives. Possible advantages of liquid formulations include ease and economy of manufacture and convenience for the end user.

Lyophilized formulations may also provide certain advantages. Potential benefits of lyophilization include improved protein stability as well as ease and economy of shipping and storage.

In addition to the choice of the basic form of the composition (e.g., lyophilized, liquid, frozen, etc.), optimization of a protein formulation typically involves varying the components of the formulation and their respective concentrations to maximize protein stability. A variety of factors may affect protein stability, including ionic strength, pH, temperature, freeze/thaw cycles, shear forces, freezing, drying, agitation, and reconstitution. Protein instability may be caused by physical degradation (e.g., denaturation, aggregation, or precipitation) or chemical degradation (e.g., deamidation, oxidation, or hydrolysis). Optimization of formulation components and concentrations may include empirical studies and/or rational approaches to overcoming sources of instability.

Accordingly, there exists a need to provide formulations that allow stable storage of a variety of proteins and that are suitable for various classes of protein pharmaceuticals, and immunoglobulin (Ig) fusion proteins, in particular.

### SUMMARY

This invention is based, at least in part, on the discovery of certain compositions containing Ig fusion proteins that are sufficiently stable during long-term storage and/or after one or more freeze/thaw cycles. The invention provides pharmaceutical compositions that contain an Ig fusion protein and at least the following four non-proteinaceous components: (1) a bulking agent, (2) a disaccharide, (3) a surfactant, and (4) a buffer. In some embodiments, the composition further contains NaCl. The compositions do not contain arginine or cysteine.

Ig fusion proteins are known in the art and are described in, e.g., U.S. Pat. Nos. 5,516,964, 5,225,538, 5,428,130, 5,514,582, 5,714,147, 5,455,165 and 6,136,310. In some embodiments, the Ig fusion protein is acidic, e.g., an Ig fusion protein having a pI of less than 6.0. In illustrative embodiments, the acidic Ig fusion proteins are PSGL-Ig, GP1b-Ig, IL-13R-Ig, and IL-21R-Ig.

In some embodiments, the Ig fusion protein is highly acidic, e.g., an Ig fusion protein having a pI of less than 4.0. In illustrative embodiments, the highly acidic Ig fusion protein is PSGL-Ig.

In some embodiments, the non-Ig portion of the Ig fusion protein is a cytokine receptor, e.g., an interleukin receptor. In illustrative embodiments, the cytokine receptors are IL-13R and IL-21R.

In some embodiments, the non-Ig portion of the Ig fusion protein is sulfated, phosphorylated, and/or glycosylated. In illustrative embodiments, the sulfated Ig fusion proteins are PSGL-Ig and GP1b-Ig. In illustrative embodiments, the glycosylated Ig fusion proteins are PSGL-Ig, GP1b-Ig, and IL-13R-Ig. In some embodiments, the glycosylated Ig fusion proteins are fucosylated and/or sialylated. In illustrative embodiments, the fucosylated Ig fusion proteins are PSGL-Ig and GP1b-Ig. In illustrative embodiments, the sialylated Ig fusion proteins are PSGL-Ig, GP1b-Ig, and IL-13R-Ig.

Illustrative examples of bulking agents include glycine and mannitol. Illustrative disaccharides include sucrose and trehalose. Illustrative examples of surfactants include polysorbate 20 and polysorbate 80. Illustrative examples of buffers include amine and phosphate buffers. Illustrative examples of amine-based buffers include histidine and tromethamine (Tris).

In some embodiments, the components of the compositions of the invention are present in defined concentration ranges. In some embodiments, the concentration of protein is from 0.025 to 60 mg/ml; the concentration of the bulking agent is from 0.5 to 5%; the concentration of disaccharide is from 0.5 to 5%; the concentration of surfactant is from 0.001 to 0.5%; all independently of each other. In some embodiments, the concentration of NaCl is from 1 to 200 mM NaCl. In certain embodiments, the concentration of NaCl is less than 35 mM. In particular embodiments, the pharmaceutical compositions includes from 1 to 4% bulking agent, from 0.5 to 2% disaccharide, and from 0.005 to 0.02% surfactant. In illustrative embodiments, the composition includes 2% bulking agent, 1% disaccharide, and 0.01% surfactant.

The invention further relates to the physical state of the formulation. The invention provides, without limitation, liquid, frozen, lyophilized, and reconstituted formulations.

The invention further provides methods of making compositions of the invention, including methods wherein the composition is lyophilized by a process that includes an annealing step.

The foregoing summary and the following detailed description are exemplary and explanatory only and are not restrictive of the invention, as claimed.

### BRIEF DESCRIPTION OF THE FIGURE

FIG. 1 shows the effect of polysorbate-80 on protein aggregation and protein recovery in GP1b-Ig formulations after up to 14 freeze/thaw cycles. GP1b-Ig was formulated at 2 mg/mL in 20 mM Tris pH 7.2, 50 mM NaCl, and 0%, 0.005%, 0.01%, or 0.02% polysorbate-80. Vials of each formulation were subjected to up to 14 cycles of freeze/thaw and assayed for % high molecular weight species (HMW) by SEC-HPLC. Protein recovery was monitored by the HPLC detector signal at 280 and 214 nm. The X axis shows number of freeze/thaw cycles. The Y axis shows percent HMW.

### DETAILED DESCRIPTION

This invention provides compositions comprising Ig fusion proteins. The invention is based, at least in part, on the discovery that compositions comprising an Ig fusion protein, a buffer, a disaccharide, a bulking agent, and a surfactant are rendered sufficiently stable for long-term storage and/or one or more freeze/thaw cycles. The invention also provides methods of preparing Ig fusion compositions.

### Ig Fusion Proteins

The invention provides compositions comprising immunoglobulin (Ig) fusion proteins.

An Ig fusion protein is a protein that comprises (a) a non-Ig portion linked to (b) an Ig portion which is derived from the constant region of an immunoglublin.

In some embodiments, the Ig fusion protein is acidic, e.g., an Ig fusion protein having a isoelectric point (pI) of less than 6.0, 5.5, 5.0, 4.5, 4.0, 3.5, 3.0, or 2.5. In illustrative embodiments, e.g., the acidic Ig fusion proteins are PSGL-Ig, GP1b-Ig, IL-13R-Ig, and IL-21R-Ig. The isoelectric point of a protein is the pH at which its net charge is zero. Methods for determining the isoelectric point of a protein of interest are well known in the art and include, but are not limited to, theoretical calculations based on the amino acid sequence of the protein and direct measurement of pI by isoelectric focusing (IEF). Skoog, B. and Wichman, A., Trends Anal. Chem. 5:82-83 (1986); Patrickios, C. S. and Yamasaki, E. N., Anal. Biochem., 231:82-91 (1995); Alberts et al., Molecular Biology of the Cell, Third Edition, p. 171 (1994).

In some embodiments, the Ig fusion protein is highly acidic, e.g., an Ig fusion protein having a pI of less than 4.0, 3.5, 3.0, or 2.5. In illustrative embodiments, the highly acidic Ig fusion protein is PSGL-Ig.

In some embodiments, the non-Ig portion of the Ig fusion protein is derived from a receptor, e.g., a cytokine receptor. In illustrative embodiments, the cytokine receptor is an interleukin receptor. In illustrative embodiments, the cytokine receptors are IL-13R and IL-21R. Other cytokine receptors may be used, e.g., cytokine receptors described in Cytokine Reference, vol. 2: Receptors, eds. Oppenheim & Feldman, Academic Press, 2001.

In some embodiments, the Ig fusion protein comprises a non-Ig portion that is sulfated, phosphorylated, and/or glycosylated. In illustrative embodiments, the sulfated Ig fusion proteins are PSGL-Ig and GP1b-Ig. In illustrative embodiments, the glycosylated Ig fusion proteins are PSGL-Ig, GP1b-Ig, and IL-13R-Ig. In some embodiments, the glycosylated Ig fusion proteins are fucosylated and/or sialylated. In illustrative embodiments, the fucosylated Ig fusion proteins are PSGL-Ig and GP1b-Ig. In illustrative embodiments, the sialylated Ig fusion proteins are PSGL-Ig, GP1b-Ig, and IL-13R-Ig. Methods for detecting and analyzing sulfation, phosphorylation, and glycosylation of proteins are well known in the art and are described in, e.g., Posttranslational Modifications of Proteins: Tools for Functional Proteomics (Methods in Molecular Biology), Christoph Kannicht Ed. (2002).

In some embodiments, the Ig portion of the Ig fusion proteins is derived from an Fc domain of an immunoglobulin, e.g., IgG (IgG₁, IgG₄, or another IgG isotype), of human, murine, or other species, and includes functional portions of naturally occurring Ig sequences, as well as mutations and modification of such sequences. Sequences of various Fc domains are well known in the art and are provided in, e.g., Sequences of Proteins of Immunological Interest, US Department of Health and Human Services, eds. Kabat et al., 1991. The Ig portion may contain any one or all of the following portion of the Fc domain: CH1, CH2, CH3, and the hinge region. For example, the Fc domain may contain CH2, CH3, and the hinge region, but not CH1.

Additionally, Ig fusion proteins may comprise a linker that joins the non-Ig and Ig portions.

In some embodiments, the concentration of an Ig fusion protein in a composition of the invention is less than 100 mg/ml, less than 90 mg/ml, less than 80 mg/ml, less than 70 mg/ml, less than 60 mg/ml, less than 50 mg/ml, less than 40 mg/ml, less than 30 mg/ml, less than 20 mg/ml, less than 15 mg/ml, less than 10 mg/ml, or less than 5 mg/ml. In some embodiments, the concentration of an Ig fusion protein in a composition of the invention is chosen from the following ranges: from about 0.025 to about 60 mg/ml, from about 0.025 to about 40 mg/ml, from about 0.025 to about 20 mg/ml, or from about 0.025 to about 10 mg/ml. In an illustrative embodiment, the concentration of Ig fusion protein is about 10 mg/ml.

### Buffers

The compositions of the invention comprise a buffer which, in part, serves to maintain pH in a desired range. Buffers suitable for use in the invention include, but are not limited to, phosphate, citrate, acetate and amine buffers. Phosphate buffers may be, e.g., potassium phosphate or sodium phosphate. Amine buffers may be, e.g., histidine, tris(hydroxymethyl)aminomethane (“tris”), or diethanolamine.

The concentration of a buffer in the compositions of the invention may be chosen from the following ranges: from about 1 to about 1000 mM, from about 1 to about 200 mM, from about 1 to about 100 mM, from about 1 to about 50 mM, from about 1 to about 40 mM, from about 1 to about 30 mM, from about 1 to about 20 mM, or from about 1 to about 10 mM. In an illustrative embodiment, the concentration of buffer in the composition is about 10 mM.

The pH of a composition of the invention may be chosen from the following ranges: from 4 to 10, from 5 to 9, preferably, from 6 to 8. Patient discomfort may be minimized by setting the pH of an injected composition at or near physiological levels. To this end, it is preferable that the pH of the pharmaceutical composition be from about 5.8 to about 8.4, or more preferably from about 6.2 to about 7.4. Routine pH adjustments inside or outside of these ranges may be necessary to improve solubility or stability of the polypeptide or other components of the composition.

### Disaccharides

The compositions of the invention further comprises a disaccharide. Preferably, the disaccharide is a non-reducing sugar, e.g., sucrose or trehalose. In certain embodiments, the concentration of disaccharide in the composition is chosen from the following ranges: from 0.5 to 5%, from 0.5 to 4%, from 0.5 to 3%, from 0.5 to 2.5%, from 0.5 to 2%, from 0.5 to 1.5%, from 0.5 to 1%, from 1 to 1.5%, from 1.5 to 2%, from 2 to 2.5%, from 2.5 to 3%, from 3 to 4%, from 4 to 5%, or more than 5%. In particular embodiments, the concentration of disaccharide in the composition is about 0.5 to 5%, for example about 0.5 to 2.0%. In illustrative embodiments, the disaccharide concentration is 0.9 or 1.0%.

In one aspect, the disaccharide serves to stabilize the protein during freezing. As protection during freezing may depend upon the absolute concentration of the disaccharide (Carpenter et al., Pharmaceutical Research 14:969-975 (1997)), concentrations greater than 5% may be necessary to maximize stability.

In one aspect, the disaccharide stabilizes the protein during drying. Protection during drying may depend upon the final mass ratio between the final mass ratio between the disaccharide and the protein. Carpenter et al., Pharmaceutical Research 14:969-975 (1997). Accordingly, in some embodiments, the concentration of disaccharide is selected to achieve the desired mass ratio of disaccharide to protein, typically at least 1:1. In some embodiments, stability is optimized at a disaccharide:protein mass ratio of about 5:1. In other embodiments, the disaccharide:protein mass ratio is 10:1, 20:1, 30:1, 40:1, 50:1, 100:1, 200:1, 300:1, 400:1, 500:1, 600:1, 700:1, 800:1, 900:1, 1000:1, or higher than 1000:1.

The disaccharide may act as a lyoprotectant or cryoprotectant. “Lyoprotectants” include substances that prevent or reduce chemical or physical instability of a protein upon lyophilization and subsequent storage. In one aspect, the lyoprotectant prevents or reduces chemical or physical instabilities in the protein as water is removed from the composition during the drying process. In a further aspect, the lyoprotectant stabilize the protein by helping maintain the proper conformation of the protein through hydrogen bonding.

“Cryoprotectants” include substances that provide stability to the frozen protein during production, freezing, storage, handling, distribution, reconstitution, or use. In a particular aspect, “cryoprotectants” include substances that protect the protein from stresses induced by the freezing process. Cryoprotectants may have lyoprotectant effects.

### Bulking Agents

The composition of the invention further comprises one or more bulking agents of the following: glycine and mannitol. The bulking agents serve to contribute to the mass and physical structure of the lyophilized cake. In one aspect, bulking agents contribute to the formation of an elegant cake. More specifically, bulking agents promote the formation of a cake that is aesthetically acceptable, uniform, or mechanically strong. Bulking agents also promote the formation of an open pore structure and the ease and speed of reconstitution. Bulking agents also reduce or prevent cake collapse, eutectic melting, or retention of residual moisture. In another aspect, bulking agents help protect the protein against stresses (e.g., physical and chemical stresses) and help maintain protein activity.

In certain embodiments, the concentration of bulking agent in the composition is chosen from the following ranges: from 0.5 to 1%, from 1 to 1.5%, from 1.5 to 2%, from 2 to 2.5%, from 2.5 to 3%, from 3 to 3.5%, from 3.5 to 4%, from 4 to 4.5%, from 4.5 to 5%, more than 5%, from 0.5 to 5%, from 0.5 to 4%, from 0.5 to 3%, from 0.5 to 2.5%, from 0.5 to 2%, from 0.5 to 1.5%, or from 0.5 to 1%. In certain embodiments, the concentration of bulking agent in the composition is 0.5 to 5%, for example 0.5 to 3%, even more precisely 1.8 to 2%.

### Surfactants

Preferably, the composition of the invention also comprises a surfactant. In one aspect, surfactants protect the protein from stresses induced at interfaces (e.g., an air/solution interface or solution/surface interface). In one embodiment, surfactants prevent or reduce aggregation. Surfactants include detergents, such as polysorbate, e.g., polysorbate-20 or polysorbate-80, and polymers, such as polyethyleneglycol. A variety of surfactants are known in the art (see, for example, U.S. Pat. No. 6,685,940, column 16, lines 10-35). In an illustrative embodiment, the surfactant is vegetable-derived polysorbate-80.

In certain embodiments, the concentration of surfactant in the composition is from 0.001 to 0.5%, from 0.001 to 0.2%, from 0.001 to 0.1%, from 0.001 to 0.05%, from 0.001 to 0.01%, or from 0.001 to 0.005%. In illustrative embodiments, the concentration of surfactant in the composition is from 0.005 to 0.01%.

### Other Components

The composition may further comprise additional pharmaceutically acceptable components. Suitable additional components include additional tonicity modifiers and other excipients known in the art.

A tonicity modifier is a substance that contributes to the osmolality of the composition. The osmolality of human serum is about 300±50 mOsM/kg. To maintain protein stability and minimize patient discomfort, it is generally preferable that the pharmaceutical composition be isotonic, i.e., having approximately equal osmolality, with human serum. Accordingly, the osmolality of the composition is preferably from 180 to 420 mOsM/kg. However, one of skill in the art will understand that the osmolality of the composition may be higher or lower as specific conditions require. A variety of tonicity modifiers are known in the art (see, e.g., paragraph 0047 of U.S. Patent Application 20030180287). Other components of the composition, including, but not limited to, buffers, disaccharides, bulking agents, and surfactants, may also contribute to the osmolality of the composition.

Excipients include, but are not limited to, chemical additives, co-solutes, and co-solvents. Preferably, excipients contribute to the stability of the protein, but it is to be understood that excipients may otherwise contribute to the physical, chemical, and biological properties of the composition. A variety of excipients are known in the art (see, e.g., paragraphs 0048-0049 of U.S. Patent Application 20030180287).

In some embodiments, the composition further comprises sodium chloride. In particular embodiments, the composition comprises 1-200 mM, or less than 50 mM, less than 40 mM, less than 35 mM, less than 30 mM, less than 25 mM, less than 20 mM, less than 15 mM, less than 10 mM, or less than 5 mM NaCl. Under certain conditions, NaCl may cause difficulty during lyophilization or lead to the appearance of opalescence in the reconstituted lyophilate. Accordingly, in a particular embodiment, the composition does not comprise NaCl.

It is to be understood that certain components of the composition may be interchanged with alternatives known in the art. However, one skilled in the art will also understand that inclusion of certain components will preclude the use of other components, concentrations, or methods of preparing the pharmaceutical composition, for reasons that include, but are not limited to, chemical compatibility, pH, tonicity, and stability.

The compositions of the invention contain no more than 0.5 mM, 0.1 mM, 0.01 mM, no detectable level, or none of any of arginine or its salt, or cysteine or its salt. These compounds are not added in preparing the composition, or cannot be detected in the composition, at more than these limits. These restrictions apply only to the free amino acids and their salts, as opposed to arginine and/or cysteine present in the polypeptide.

### Illustrative Embodiments

In some embodiments, the pharmaceutical composition consists essentially of an Ig fusion protein, a buffer, a disaccharide, a bulking agent, and a surfactant. In some embodiments, the pharmaceutical composition comprises an Ig fusion protein, a buffer, a disaccharide, a bulking agent, and a surfactant.

In one illustrative embodiment, the pharmaceutical composition comprises a pharmaceutically effective amount of an Ig fusion protein, from 1 mM to 1 M buffer, from 0.5 to 5% disaccharide, from 0.5 to 5% bulking agent, and from 0.001 to 0.5% surfactant. In a further embodiment, the pharmaceutical composition comprises a pharmaceutically effective amount of an Ig fusion protein, from 1 mM to 1 M buffer, from 0.5 to 5% disaccharide, from 0.5 to 5% bulking agent, from 0.001 to 0.5% surfactant, and from 1 to 200 mM NaCl. In a further embodiment, the pharmaceutical composition comprises a pharmaceutically effective amount of an Ig fusion protein, from 1 mM to 1 M buffer, from 0.5 to 5% disaccharide, from 0.5 to 5% bulking agent, from 0.001 to 0.5% surfactant, and less than 35 mM NaCl. In a further embodiment, the pharmaceutical composition comprises a pharmaceutically effective amount of an Ig fusion protein, from 1 mM to 1 M buffer, from 0.5 to 5% disaccharide, from 0.5 to 5% bulking agent, and from 0.001 to 0.5% surfactant, and does not contain NaCl.

In one illustrative embodiment, the pharmaceutical composition comprises from 0.025 to 20 mg/ml Ig fusion protein, from 5 to 30 mM buffer, from 0.5 to 2% disaccharide, from 1.5 to 2.5% bulking agent, and from 0.001 to 0.02% surfactant.

In a further embodiment, the pharmaceutical composition comprises about 10 mg/ml Ig fusion protein, about 10 mM buffer, from about 1.8 to about 2% bulking agent, from about 0.9 to about 1% disaccharide, and from about 0.005 to about 0.01% surfactant.

In a further embodiment, the pharmaceutical composition comprises about 10 mg/ml Ig fusion protein, about 10 mM buffer, from about 1.8 to about 2% glycine, from about 0.9 to about 1% disaccharide, and from about 0.005 to about 0.01% surfactant.

In a further embodiment, the pharmaceutical composition comprises about 10 mg/ml Ig fusion protein, about 10 mM buffer, from about 1.8 to about 2% mannitol, from about 0.9 to about 1% disaccharide, from about 0.005 to about 0.01% surfactant, and less than 35 mM NaCl.

In one illustrative embodiment, the pharmaceutical composition comprises 10 mg/ml PSGL-Ig, 10 mM histidine, 260 mM glycine, 10 mM NaCl, 1% sucrose, and 0.005% polysorbate-80.

In one illustrative embodiment, the pharmaceutical composition comprises 10 mg/ml GP1b-Ig, 10 mM histidine, 1.8% glycine, 25 mM NaCl, 0.9% sucrose, and 0.01% polysorbate-80.

In one illustrative embodiment, the pharmaceutical composition comprises 10 mg/ml IL-13R-Ig, 10 mM Tris, 2% mannitol, 40 mM NaCl, 1% sucrose, and 0.01% polysorbate-80.

### Physical State of the Composition

A variety of physical states are suitable for the pharmaceutical composition of the invention, including, but not limited to, liquid, frozen liquid, lyophilized, and reconstituted formulations. Reconstituted formulations include lyophilized compositions that have been resuspended in liquid, typically water for injection (WFI). For lyophilized compositions, concentrations and osmolalities typically refer to those of the pre-lyophilized liquid composition, although these values may alternatively refer to the reconstituted composition. For frozen liquid compositions, concentrations and osmolalities typically refer to the liquid composition prior to freezing.

### Methods of Preparing Pharmaceutical Compositions

One skilled in the art will know a variety of methods suitable for preparing the pharmaceutical composition of the invention. The skilled artisan will also understand that some components may interact in such a way as to make certain methods or orders of preparation unfavorable.

In one embodiment, the composition is prepared by exchanging purified protein into a solution comprising all of the remaining components of the composition except the surfactant and subsequently adding the surfactant to the desired concentration.

In one embodiment, the composition is lyophilized by a process that includes an annealing step, i.e., holding the composition at a temperature above the final freezing temperature for a defined period to promote crystallization of the potentially crystalline components. In one aspect, annealing allows complete or more thorough crystallization of the bulking agent, which may improve cake structure or protein stability. Further, crystallization of the bulking agent can increase the T_{g}′ of the amorphous phase, which can facilitate more efficient drying by allowing primary drying to be performed at a higher temperature, again resulting in improved cake quality or stability. See Wang, International Journal of Pharmaceutics 203:1-60 (2000). Further, failure to completely crystallize the bulking agent may allow crystallization during primary drying, which can lead to vial breakage (Tang and Pikal, Pharmaceutical Research 21:191-200 (2004), or crystallization during storage, which can destabilize the protein (Carpenter, et al., Pharmaceutical Research 14:969-975 (1997)).

In an illustrative embodiment, the pharmaceutical composition is lyophilized by a process comprising the following steps: freezing the composition at less than −40° C.; annealing at a temperature between −5° C. and −40° C. for a period of time sufficient to promote crystallization of the bulking agent; lowering the temperature below −35° C.; establishing a vacuum; and drying the composition at a temperature between −20° C. and +30° C.

### Stability

In one aspect, the invention relates to stable pharmaceutical compositions. A “stable” composition is one in which the protein therein essentially retains certain physical and chemical properties upon storage or use. In one aspect, “storage or use” includes one or more cycles of freeze/thaw. Various assays of protein stability and/or instability are described in the Example and other suitable methods are well known in the art and reviewed in, e.g, Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Such assays include, but are not limited to, quantification of high molecular weight material (e.g., aggregates), quantification of low molecular weight material (e.g., degradants), quantification of protein concentration, quantification of protein activity, and quantification of post-translational amino acid modifications. The pharmaceutical composition of the invention preferably contains less than 10%, less than 5%, less than 4%, less than 3%, less than 2%, less than 1%, or less than 0.5% aggregant (high molecular weight) or degradant (low molecular weight) material upon storage or use. Similarly, the pharmaceutical composition of the invention preferably retains 80%, 90%, 95%, 96%, 97%, 98%, 99%, 99.5%, or 100% of protein activity upon storage or use.

In some embodiments, the composition is stable at a certain temperature (e.g., −80° C. to 40° C.-40° C. to 40° C., at about 20° C.) for a specified time period (e.g., 1, 4, 7, 12 or 24 weeks; or 1, 2, 3, 4, 6, 7.5, 9, or 12 months; or more).

In some embodiments, the composition is stable after a specified number of freeze/thaw cycles (e.g., 1, 2, 3, 4, 5, 6, 7, 8, 9, 10, 20, or more).

In one aspect, the pharmaceutical composition is stable protein through any or all of the manufacture, shipping, and handling steps, which may include preparation of the composition, freezing, drying, storage, shipping, reconstitution, freeze/thaw cycles, and post-reconstitution storage by the end user.

A variety of factors may affect protein stability, including ionic strength, pH, temperature, freeze/thaw cycles, shear forces, freezing, drying, agitation, and reconstitution. Protein instability may be caused by physical degradation (e.g., denaturation, aggregation, or precipitation) or chemical degradation (e.g., deamidation, oxidation, or hydrolysis).

### EXAMPLES

### Example 1

### PSGL-Ig Formulation

A. PSGL-Ig background

P-selectin glycoprotein ligand-1 (PSGL-1) is the major high affinity receptor for P-selectin on human leukocytes. PSGL-Ig is a fusion protein comprising soluble PSGL linked to a mutated human IgG1 Fc domain, as described in U.S. Pat. No. 5,827,817. Isoelectric focusing (IEF) of PSGL-Ig shows predominant bands within a pH range from 2.8 to 3.3, clustered around a pI of approximately 3. Post-translational modifications of PSGL-Ig include sulfation and glycosylation. Glycosylation of PSGL-Ig includes O-linked and N-linked glycans. O-linked glycans in PSGL-Ig include sialylated and/or fucosylated structures. For a review of post-translation modifications of PSGL and their biological significance, see Liu et al., Journal of Biological Chemistry 273: 7078-7087 (1998).

B. Optimization of PSGL-Ig formulation

PSGL-Ig was purified over a QAE column and formulated at about 25 μg/ml in PBS-CMF or “His/Suc/Gly” (10 mM histidine, 260 mM glycine, 1% sucrose). Samples were processed either without polysorbate-80, with polysorbate-80 added to 0.005% prior to freeze/thaw cycling, or with polysorbate-80 added to 0.005% after freeze/thaw cycling but prior to HMW measurement (for PBS-CMF only). Samples were subjected to 20 cycles of freeze/thaw. Percent high molecular weight species (HMW) was determined before and after freeze/thaw cycling by size exclusion chromatography (SEC)-HPLC. As shown in Table 1, polysorbate-80 significantly reduced the accumulation of HMW in both PBS-CMF and His/Suc/Gly, but only when added prior to freeze/thaw cycling.

TABLE 1
Optimization of PSGL-lg Formulation

| | | % HMW at | % HMW after |
|---|---|---|---|
| Buffer | Polysorbate addition | Time Zero | 20X Freeze/Thaw |
| PBS-CMF | none | 4.25 | 46.52 |
| PBS-CMF | spiked in post-F/T | 5.28 | 44.18 |
| PBS-CMF | 0.005% | 4.18 | 4.86 |
| His/Suc/Gly | 0.005% | 4.39 | 3.37 |
| His/Suc/Gly | none | | 42.97 |

To determine the contribution of the buffering agent and pH to PSGL-Ig stability, PSGL-Ig was formulated at about 50 μg/ml in 15 mM buffer alone (without additional excipients). Five buffers (succinate, citrate, histidine, phosphate, and Tris) were tested at a total of 7 pH's (see Table 2). Samples were subjected to 5 cycles of freeze/thaw. Percent HMW was determined before and after freeze/thaw cycling by SEC-HPLC. As shown in Table 2, citrate and histidine resulted in minimal changes in percent HMW after freeze/thaw, while tris, phosphate, and succinate resulted in higher percent increases in HMW.

TABLE 2
Effect of PSGL-lg Formulation pH on HMW Accumulation

| Buffer | pH | increase in HMW |
|---|---|---|
| succinate | 5.44 | 11.7% |
| citrate | 5.94 | 1.8% |
| citrate | 6.52 | 1.4% |
| histidine | 6.67 | 2.7% |
| phosphate | 7.02 | 6.6% |
| phophate | 7.58 | 3.1% |
| Tris | 7.51 | 3.5% |

### C. Long-Term Stability of PSGL-Ig Formulation

Purified PSGL-Ig was exchanged into 1% sucrose, 260 mM glycine, 10 mM NaCl, and 10 mM histidine, pH 6.5-6.6 at 5 mg/mL. Polysorbate-80 was added to a final concentration of 0.005%. 1 ml aliquots were filled into Type I glass 2 ml tubing vials and stoppered. Samples were either lyophilized and stored at 5° C., 25° C., or 40° C., or stored as a frozen liquid at −80° C. Protein stability was assessed by HMW formation, degree of hyposulfation, biological activity measured in relative binding units (RBU), and degree of cyclization of the N-terminal glutamines to pyro-glutamic acid. Time zero samples consisted of pre- and post-lyophilization formulations. Samples were also analyzed after 3 or 7.5 months of storage at the temperatures listed above. As shown in Table 3, the lyophilized and frozen liquid formulations are stable under the conditions tested.

TABLE 3
Stability of PSGL-lg Formulation
HMW by SEC (as area %)

| | liquid | lyo, time zero | | |
|---|---|---|---|---|
| Temperature | 0.53 | 0.52 | 3 months | 7.5 months |
| −80° C. | | | | 0.63 |
| 5° C. | | | 0.27 | 0.52 |
| 25° C. | | | 0.39 | 0.71 |
| 40° C. | | | 0.38 | |

RBU (biological activity)

| | liquid | lyo, time zero | | |
|---|---|---|---|---|
| Temperature | 0.99 | 1.1 | 3 months | 7.5 months |
| −80° C. | | | | 0.81 |
| 5° C. | | | 1.6 | 0.85 |
| 25° C. | | | 1.3 | 0.96 |
| 40° C. | | | 1.2 | |

Ratio Q-Q/<Q-<Q (fully uncyclized to fully cyclized)

| | liquid | lyo, time zero | | |
|---|---|---|---|---|
| Temperature | 1.68 | 1.64 | 3 months | 7.5 months |
| −80° C. | | | | nd |
| 5° C. | | | 1.67 | 1.78 |
| 25° C. | | | 1.64 | 1.72 |
| 40° C. | | | 1.56 | |

Hyposulfation (by AEX)

| | % hyposulfated |
|---|---|
| Temperature | at 7.5 months |
| −80° C. | 1.06 |
| 5° C. | 1.00 |
| 25° C. | 1.00 |

### Example 2

### GP1b-Ig Formulation

A. GP1b-Ig background

GP1b-alpha is a receptor expressed by platelets. The major ligand for GP1b-alpha is von Willebrand Factor (VWF). GP1b-Ig is a fusion protein comprising the soluble N-terminal 290-amino acid ligand-binding domain of GP1b-alpha linked to 225 amino acids of an inactivated human IgG1 Fc, as described in PCT Publication Number WO 02/063003. The VWF-binding domain of GP1b-Ig comprises two gain-of-function mutations, M239V and G233V, which enhance its VWF binding affinity. Isoelectric focusing (IEF) of GP1b-Ig shows predominant bands within a pH range from 4.1 to 5.6, clustered around a pI of approximately 5. Post-translational modifications of GP1b-Ig include N-linked glycosylation. N-linked glycans in GP1b-Ig include sialylated and/or fucosylated structures.

B. Optimization of GP1b-Ig Formulation

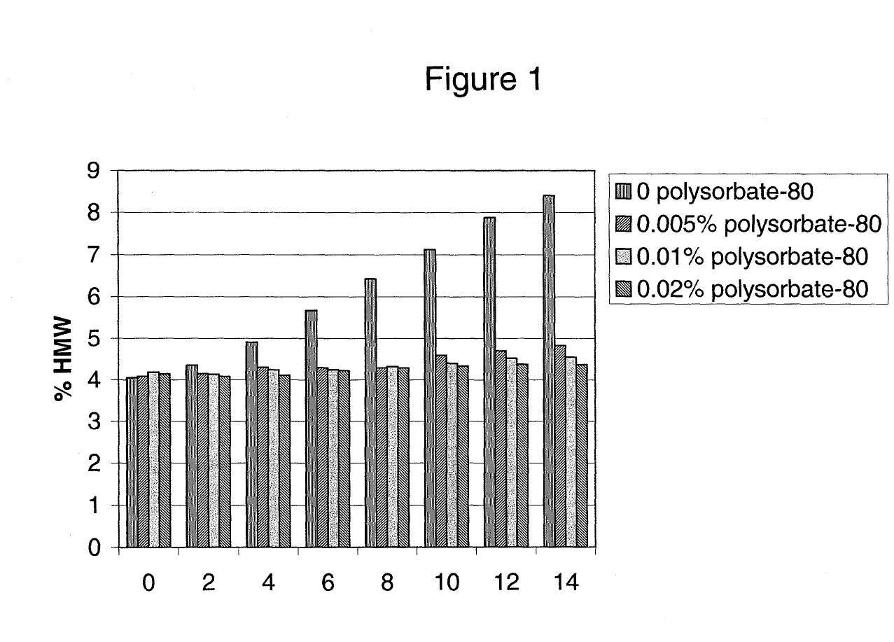

The effect of polysorbate-80 on GP1b-Ig stability was evaluated in 2 ml polypropylene vials. GP1b-Ig was formulated at 2 mg/ml in 20 mM Tris pH 7.2, 50 mM NaCl, and varying concentrations of polysorbate-80 (0%, 0.005%, 0.01%, or 0.02%). Vials were frozen by submerging in liquid nitrogen for 1 minute and thawed by incubation in a 20° C. water bath until no ice remained. Samples were subjected to 14 freeze/thaw cycles, with aliquots withdrawn for analysis after every other cycle. Percent HMW was determined by SEC-HPLC. Protein recovery was evaluated by integrating the area of protein peaks on an absorbance signal at 280 nm. As shown in FIG. 1, addition of polysorbate-80 significantly reduced HMW accumulation.

The effect of glycine on GP1b-Ig stability was evaluated in a similar manner. GP1b-Ig was formulated at 5 mg/ml in 10 mM histidine pH 6.5, 25 mM NaCl, 0.9% sucrose, 0.01% polysorbate-80, and varying concentrations of glycine (0%, 1.0%, 1.8%, 2.0%, or 4.0% w/v). Vials were frozen by submerging in liquid nitrogen for 1 minute and thawed by incubation in a 20° C. water bath until no ice remained. Samples were subjected to 10 freeze/thaw cycles, with aliquots withdrawn for analysis after every other cycle. Percent HMW was determined by SEC-HPLC. As shown in Table 4, addition of 1.8% glycine minimized HMW accumulation after 6, 8, or 10 freeze/thaw cycles.

TABLE 4
Effect of Glycine Concentration on % HMW in GP1b-Ig Formulations

Number of Freeze/Thaw Cycles

| % Glycine | 0 | 2 | 4 | 6 | 8 | 10 |
|---|---|---|---|---|---|---|
| 0 | 0.35 | 0.41 | 0.37 | 0.44 | 0.47 | 0.52 |
| 1 | 0.37 | 0.36 | 0.4 | 0.44 | 0.46 | 0.47 |
| 1.8 | 0.38 | 0.39 | 0.38 | 0.4 | 0.42 | 0.46 |
| 2 | 0.35 | 0.39 | 0.37 | 0.42 | 0.44 | 0.48 |
| 4 | 0.4 | 0.38 | 0.41 | 0.45 | 0.52 | 0.59 |

The effect of pH on GP1b-Ig stability was evaluated in similar manner. GP1 b-Ig was formulated at 1 mg/ml in 20 mM sodium phosphate at varying pH levels (5.0, 6.0, 7.0, or 8.0). Vials were frozen by submerging in liquid nitrogen for 1 minute and thawed by incubation in a 20° C. water bath until no ice remained. Samples were subjected to 10 freeze/thaw cycles, with aliquots withdrawn for analysis after every other cycle. Percent HMW was determined by SEC-HPLC and protein recovery was monitored by the HPLC detector signal at 280 and 214 nm. After 10 cycles, samples at pH 5.0 and 6.0 became cloudy, an indication of declining recovery of soluble protein. As shown in Table 5, formulation at pH 5.0 or 6.0 leads to increased HMW accumulation and decreased protein recovery.

TABLE 5
Effect of pH on % HMW and Protein Recovery in GB1b-Ig Formulations

| | | Number of Freeze/Thaw Cycles | | | | | |
|---|---|---|---|---|---|---|---|
| | pH | 0 | 2 | 4 | 6 | 8 | 10 |
| % HMW | 5.0 | 4.62 | 9.55 | 12.02 | 14.45 | 19.33 | 24.94 |
| | 6.0 | 3.96 | 8.63 | 12.33 | 15.93 | 20.64 | 24.94 |
| | 7.0 | 4.44 | 6.53 | 8.6 | 10.17 | 12.02 | 13.67 |
| | 8.0 | 4.69 | 6.32 | 7.48 | 8.79 | 10.31 | 11.77 |
| Protein | 5.0 | 9.10E+05 | 8.30E+05 | 7.38E+05 | 6.17E+05 | 5.03E+05 | 4.06E+05 |
| Recovery | 6.0 | 8.93E+05 | 8.69E+05 | 8.26E+05 | 7.96E+05 | 7.63E+05 | 7.37E+05 |
| (a.u.) | 7.0 | 9.23E+05 | 9.12E+05 | 9.00E+05 | 8.88E+05 | 8.77E+05 | 8.60E+05 |
| | 8.0 | 9.37E+05 | 9.32E+05 | 9.28E+05 | 9.11E+05 | 9.00E+05 | 8.91E+05 |

The effect of protein concentration on GP1b-Ig stability was evaluated in 20 ml bottles, half filled with 0.25, 10, or 19 mg/ml GP1b-Ig in 10 mM histidine pH 6.5, 1.8% glycine, 25 mM NaCl, 0.9% sucrose, and 0.01% polysorbate-80. The bottles were frozen by submerging in liquid nitrogen for 10 minutes and thawed by incubation in a 25° C. water bath for 15-20 minutes. Samples were subjected to 10 cycles of freeze/thaw, with aliquots removed for analysis after 0, 1, 2, 4, 6, 8, and 10 cycles. Prior to analysis, the protein concentration in all samples was normalized by dilution to 0.25 mg/ml in an otherwise identical formulation. Percent HMW was determined by SEC-HPLC. As shown in Table 6, the GP1b-Ig is stable over a broad range of concentrations in this formulation.

TABLE 6
Effect of Protein Concentration on % HMW in GP1b-Ig Formulations

| | |
|---|---|
| [GP1b-Ig], | Number of Freeze/Thaw Cycles |

| in mg/ml | 0 | 2 | 4 | 6 | 8 | 10 |
|---|---|---|---|---|---|---|
| 19 | 0.62 | 0.67 | 0.42 | 0.54 | 0.56 | 0.59 |
| 10 | | 0.58 | 0.51 | 0.40 | 0.55 | 0.32 |
| 0.25 | | 0.42 | 0.40 | 0.36 | 0.35 | 0.46 |

C. Long-Term Stability of GP1b-Ig Formulation

Purified GP1b-Ig was stored at −80° C. until thawed for use. Protein concentration was estimated to be 19 mg/ml by A280. GP1b-Ig was formulated to 10 mg/ml in 10 mM histidine pH 6.5, 1.8% glycine, 25 mM NaCl, 0.9% sucrose, and 0.01% polysorbate-80 by dilution in the appropriate stock solution. The resulting formulation was filtered through a 0.2 μm filter unit and dispensed into glass vials. Vials were placed on steel trays and lyophilized. Lyophilization was performed according to the cycle parameters summarized in Table 7.

TABLE 7
GP1b-lg Lyophilization Cycle

| Temp (° C.) | Time (hrs) | Ramp (hrs) | Pressure (mT) |
|---|---|---|---|
| 5 | 13.5 | 1.8 | |
| −50 | 4 | 1.2 | |
| −15 | 6 | 0.8 | |
| −40 | 1.5 | 0.5 | 50 (last hour only) |
| −25 | 35.3 | 4.6 | 50 |
| 30 | 6 | | 50 |
| Total Hours | 75.2 | | |

Upon completion of the cycle, the lyophilization chamber was back-filled with dry nitrogen, after which the stoppers were depressed and the remaining vacuum released. The vials were immediately crimped, labeled, and stored at 2-8° C., 25° C., or 40° C.

At each time point (0, 1, 2, 3, 4, 6, 9, and 12 months), one or more lyophilization vials were opened. The product cakes were assessed for appearance and residual moisture was determined by Karl-Fisher titration. Cakes were then reconstituted with 1 ml water for injection (WFI), monitoring for appearance and reconstitution time. After reconstitution, pH was measured by dipping the electrode into a 2 ml cryo vial containing 400 μl of the formulation. The remaining solution was divided into 3×200 μl aliquots, two of which were frozen at −80° C., and one of which was designated for analysis and kept at 2-8° C.

The reconstituted GP1b-Ig formulations were diluted 10 times in the same solution to theoretically yield a 1 mg/ml solution. Actual protein concentrations were measured by UV spectroscopy using UV-transparent 96-well plates. Protein concentration was calculated by the formula: concentration (in mg/ml)=dilution factor×(A280-A320)/(1.1).

The biological activity was assessed as degree of binding of GP1b-Ig to human, plasma-derived VWF. The measured protein concentration was used to calculate specific activity in units/μg.

HMW accumulation was measured by SEC-HPLC and was expressed as a percentage of total species.

Accumulation of low molecular weight species (LMW) was monitored by reverse phase (RP)-HPLC and expressed as a percentage of total species.

The distribution of sulfated isoforms was determined by anion-exchange chromatography (AEX).

As shown in Tables 8 through 10, these assays demonstrate that this GP1b-Ig formulation is stable under the conditions tested for at least 9 months. Table 8 shows results obtained following storage at 2-8° C. Table 9 shows results obtained following storage at 25° C. Table 10 shows results obtained following storage at 40° C.

TABLE 8
Stability of GP1b-Ig Formulation at 2-8° C.

| | | | Protein | % | VWF | | | |
|---|---|---|---|---|---|---|---|---|
| | | | Conc. | Residual | Binding | % | % | |
| | Appearance | pH | (mg/ml) | Moisture | Activity | HMW | LMW | Sulfation |
| Specification | Before | 6.0 to 7.0 | 10 mg/ml | Report | 5.0 × 10⁴-1.8 × | ≦6% | ≦12% | |
| | Reconstitution: | | | Result | 10⁵ units/μg | | | |
| | White Cake | | | | of | | | |
| | essentially free | | | | GP1b-Ig | | | |
| | of particles | | | | | | | |
| | After | | | | | | | |
| | Reconstitution: | | | | | | | |
| | Solution | | | | | | | |
| | essentially free | | | | | | | |
| | of particles | | | | | | | |
| Initial | Meets | 6.68 | 9.99 | 0.34 | 9.4 × 10⁴ | 0.35 | 2.35 | 4.90 |
| | Specification | | | | | | | |
| 1 Month | Meets | N/A | 9.60 | 0.41 | 8.21 × 10⁴ | 0.46 | 2.82 | 4.93 |
| | Specification | | | | | | | |
| 2 Months | Meets | N/A | 8.47 | 0.54 | 7.28 × 10⁴ | 0.38 | 2.40 | 4.88 |
| | Specification | | | | | | | |
| 3 Months | Meets | N/A | 9.62 | 0.60 | 7.53 × 10⁴ | 0.45 | 2.19 | 4.87 |
| | Specification | | | | | | | |
| 4 Months | Meets | 6.38 | 9.72 | 0.35 | 1.05 × 10⁵ | 0.32 | 2.17 | 4.88 |
| | Specification | | | | | | | |
| 6 Months | Meets | 6.65 | 9.93 | 0.53 | 1.01 × 10⁵ | 0.37 | 2.49 | 4.42 |
| | Specification | | | | | | | |
| 9 Months | Meets | 6.65 | 9.64 | 0.84 | 1.07 × 10⁵ | 0.48 | 2.44 | 4.89 |
| | Specification | | | | | | | |
| 12 Months | Meets | 6.60 | 9.97 | 0.83 | 1.04 × 10⁵ | 0.48 | 2.47 | 4.88 |
| | Specification | | | | | | | |

TABLE 9
Stability of GP1b-Ig Formulation at 25° C.

| | | | Protein | % | VWF | | | |
|---|---|---|---|---|---|---|---|---|
| | | | Conc. | Residual | Binding | % | % | |
| | Appearance | pH | (mg/ml) | Moisture | Activity | HMW | LMW | Sulfation |
| Specification | Before | 6.0 to 7.0 | 10 mg/ml | Report | 5.0 × 10⁴-1.8 × | ≦6% | ≦12% | |
| | Reconstitution: | | | Result | 10⁵ units/μg | | | |
| | White Cake | | | | of | | | |
| | essentially free | | | | GP1b-Ig | | | |
| | of particles | | | | | | | |
| | After | | | | | | | |
| | Reconstitution: | | | | | | | |
| | Solution | | | | | | | |
| | essentially free | | | | | | | |
| | of particles | | | | | | | |
| Initial | Meets | 6.68 | 9.99 | 0.34 | 9.4 × 10⁴ | 0.35 | 2.35 | 4.90 |
| | Specification | | | | | | | |
| 1 Month | Meets | N/A | 9.42 | 0.91 | 8.09 × 10⁴ | 0.53 | 2.62 | 4.93 |
| | Specification | | | | | | | |
| 2 Months | Meets | N/A | 8.85 | 0.89 | 7.44 × 10⁴ | 0.46 | 2.43 | 4.89 |
| | Specification | | | | | | | |
| 3 Months | Meets | N/A | 9.67 | 1.24 | 8.36 × 10⁴ | 0.56 | 2.22 | 4.86 |
| | Specification | | | | | | | |
| 4 Months | Meets | 6.38 | 9.62 | N/A | 1.21 × 10⁵ | 0.47 | 2.42 | 4.86 |
| | Specification | | | | | | | |
| 6 Months | Meets | 6.65 | 9.49 | 1.77 | 1.03 × 10⁵ | 0.59 | 2.41 | 4.89 |
| | Specification | | | | | | | |
| 9 Months | Meets | 6.64 | 9.50 | 1.90 | 1.15 × 10⁵ | 0.69 | 2.42 | 4.89 |
| | Specification | | | | | | | |
| 12 Months | Meets | 6.60 | 10.09 | 1.40 | 9.82 × 10⁴ | 0.67 | 2.45 | 4.88 |
| | Specification | | | | | | | |

TABLE 10
Stability of GP1b-Ig Formulation at 40° C.

| | | | Protein | % | VWF | | | |
|---|---|---|---|---|---|---|---|---|
| | | | Conc. | Residual | Binding | % | % | Average |
| | Appearance | pH | (mg/ml) | Moisture | Activity | HMW | LMW | Sulfation |
| Specification | Before | 6.0 to | 10 mg/ml | Report | 5.0 × 10⁴-1.8 × | ≦6% | ≦12% | |
| | Reconstitution: | 7.0 | | Result | 10⁵ units/μg | | | |
| | White Cake | | | | of | | | |
| | essentially free | | | | GP1b-Ig | | | |
| | of particles | | | | | | | |
| | After | | | | | | | |
| | Reconstitution: | | | | | | | |
| | Solution | | | | | | | |
| | essentially free | | | | | | | |
| | of particles | | | | | | | |
| Initial | Meets | 6.68 | 9.99 | 0.34 | 9.4 × 10⁴ | 0.35 | 2.35 | 4.90 |
| | Specification | | | | | | | |
| 1 Month | Meets | N/A | 9.44 | 1.62 | 7.60 × 10⁴ | 0.68 | 2.71 | 4.93 |
| | Specification | | | | | | | |
| 2 Months | Meets | N/A | 8.42 | 1.32 | 7.91 × 10⁴ | 0.64 | 2.37 | 4.89 |
| | Specification | | | | | | | |
| 3 Months | Meets | N/A | 9.65 | 1.37 | 7.54 × 10⁴ | 0.76 | 2.21 | 4.86 |
| | Specification | | | | | | | |
| 4 Months | Meets | 6.38 | 8.79 | 1.38 | 9.49 × 10⁴ | 0.79 | 2.42 | 4.86 |
| | Specification | | | | | | | |
| 6 Months | Meets | 6.65 | 9.13 | 2.89 | 9.95 × 10⁴ | 1.12 | 2.41 | 4.89 |
| | Specification | | | | | | | |
| 9 Months | Meets | 6.64 | 9.60 | 3.18 | 1.11 × 10⁵ | 1.49 | 2.57 | 4.90 |
| | Specification | | | | | | | |
| 12 Months | Meets | 6.60 | 9.74 | 4.46 | 9.42 × 10⁴ | 1.75 | 2.55 | 4.88 |
| | Specification | | | | | | | |

### Example 3

### IL-13R-Ig Formulation

A. IL-13R-Ig background

IL-13R is the major receptor for interleukin-13 (IL-13). IL-13R-Ig is a fusion protein comprising soluble extracellular domain of IL-13Rα2 linked to a spacer sequence and the hinge CH2 CH3 regions of human IgG1 as described in U.S. Pat. No. 6,268,480. Isoelectric focusing (IEF) of IL-13R-Ig shows predominant bands within a pH range from 3.8 to 4.7, clustered around a pI of approximately 4.3. Post-translational modifications of IL-13R-Ig include N-linked glycosylation. N-linked glycans in IL-13R-Ig include sialylated structures.

### B. Optimization of IL-13R-Ig Formulation

IL-13R-Ig was formulated at 10 mg/ml in four different formulations: 10 mM NaPO₄, 0.01% polysorbate-80, 1% sucrose, and 2% mannitol, pH 7.4; 10 mM NaPO₄, 0.01% polysorbate-80, 0.9% sucrose, and 1.8% glycine, pH 7.4; 10 mM Tris, 0.01% polysorbate-80, 1% sucrose, and 2% mannitol, pH 7.4; or 10 mM Tris, 0.01% polysorbate-80, 0.9% sucrose, and 1.8% glycine, pH 7.4. Lyophilized vials were stored for up to 12 weeks at 4° C., 25° C., and 40° C. At each time point, one or more vials of each formulation and temperature combination were reconstituted and assayed for protein recovery (by A280 or SEC), HMW accumulation (by SEC-HPLC), or biological activity (by IC50 in an assay for inhibition of proliferation of an IL-13-dependent cell line). Table 11 shows the percent protein recovery, as assessed by A280, for each formulation and temperature after 12 weeks in storage. Table 12 shows the percent protein recovery, as assessed by SEC, for each formulation and temperature after 12 weeks in storage. Table 13 shows the percent HMW accumulation, as assessed by SEC-HPLC, for each combination of formulation (of the four listed above), temperature (at 4° C., 25° C., or 40° C.), and storage time (0, 1 month, 7 weeks, or 12 weeks), as well as the post-lyophilization starting material. Table 14 shows IC50 data for each formulation after 4 weeks (at 2-8° C. or 25° C.), 7 weeks (at 2-8° C. or 25° C.), or 12 weeks (at 2-8° C., 25° C., or 40° C.), as well as the post-lyophilization starting material.

TABLE 11
Effect of IL-13R-lg Formulation on % Protein
Recovery (by A280) at 12 weeks

| | Temperature | | |
|---|---|---|---|
| Formulation | 4° C. | 25° C. | 40° C. |
| 10 mM Tris, 0.01% polysorbate-80, 1% | 96.21 | 94.30 | 90.18 |
| sucrose, and 2% mannitol, pH 7.4 | | | |
| 10 mM NaPO₄, 0.01% polysorbate-80, 1% | 95.89 | 106.54 | 95.37 |
| sucrose, and 2% mannitol, pH 7.4 | | | |
| 10 mM Tris, 0.01% polysorbate-80, 0.9% | 99.08 | 104.65 | 104.08 |
| sucrose, and 1.8% glycine, pH 7.4 | | | |
| 10 mM NaPO₄, 0.01% polysorbate-80, 0.9% | 108.38 | 105.26 | 108.06 |
| sucrose, and 1.8% glycine, pH 7.4 | | | |

TABLE 12
Effect of IL-13R-lg Formulation on %
Protein Recovery (by SEC) at 12 weeks

| | Temperature | | |
|---|---|---|---|
| Formulation | 4° C. | 25° C. | 40° C. |
| 10 mM Tris, 0.01% polysorbate-80, 1% | 94 | 95 | 89 |
| sucrose, and 2% mannitol, pH 7.4 | | | |
| 10 mM NaPO₄, 0.01% polysorbate-80, 1% | 93 | 104 | 91 |
| sucrose, and 2% mannitol, pH 7.4 | | | |
| 10 mM Tris, 0.01% polysorbate-80, 0.9% | 100 | 105 | 104 |
| sucrose, and 1.8% glycine, pH 7.4 | | | |
| 10 mM NaPO₄, 0.01% polysorbate-80, 0.9% | 104 | 100 | 102 |
| sucrose, and 1.8% glycine, pH 7.4 | | | |

TABLE 13
Effect of IL-13R-Ig Formulation on % HMW (by SEC)

| | | 4° C. | | | 25° C. | | | 40° C. | | |
|---|---|---|---|---|---|---|---|---|---|---|
| Formulation | post-lyo | 4 wk | 7 wk | 12 wk | 4 wk | 7 wk | 12 wk | 4 wk | 7 wk | 12 wk |
| 10 mM Tris, | 1.87 | 1.84 | 2.28 | 1.49 | 2.19 | 2.82 | 2.00 | 2.68 | 3.71 | 2.88 |
| 0.01% | | | | | | | | | | |
| polysorbate-80, | | | | | | | | | | |
| 1% sucrose, and | | | | | | | | | | |
| 2% mannitol, pH | | | | | | | | | | |
| 7.4 | | | | | | | | | | |
| 10 mM NaPO₄, | 3.02 | 2.34 | 2.91 | 2.03 | 2.91 | 3.89 | 2.97 | 3.57 | 5.34 | 4.57 |
| 0.01% | | | | | | | | | | |
| polysorbate-80, | | | | | | | | | | |
| 1% sucrose, and | | | | | | | | | | |
| 2% mannitol, pH | | | | | | | | | | |
| 7.4 | | | | | | | | | | |
| 10 mM Tris, | 2.18 | 1.86 | 2.33 | 1.62 | 2.24 | 2.80 | 2.04 | 2.57 | 3.79 | 3.04 |
| 0.01% | | | | | | | | | | |
| polysorbate-80, | | | | | | | | | | |
| 0.9% sucrose, | | | | | | | | | | |
| and 1.8% | | | | | | | | | | |
| glycine, pH 7.4 | | | | | | | | | | |
| 10 mM NaPO₄, | 2.91 | 2.56 | 3.22 | 2.31 | 2.88 | 4.10 | 3.03 | 3.90 | 5.87 | 4.97 |
| 0.01% | | | | | | | | | | |
| polysorbate-80, | | | | | | | | | | |
| 0.9% sucrose, | | | | | | | | | | |
| and 1.8% | | | | | | | | | | |
| glycine, pH 7.4 | | | | | | | | | | |

TABLE 14
Effect of IL-13R-Ig Formulation on Bioactivity (IC50 in pM)

| | | 4 weeks | | 7 weeks | | 12 weeks | | |
|---|---|---|---|---|---|---|---|---|
| Formulation | post-lyo | 2-8° C. | 25° C. | 2-8° C. | 25° C. | 2-8° C. | 25° C. | 40° C. |
| 10 mM NaPO₄, | 863 | 539 | 672 | 727 | 1034 | ND | ND | ND |
| 0.01% polysorbate- | | | | | | | | |
| 80, 0.9% sucrose, | | | | | | | | |
| and 1.8% glycine, pH | | | | | | | | |
| 7.4 | | | | | | | | |
| 10 mM Tris, 0.01% | 1014 | 904 | 637 | 632 | 818 | 629 | 865 | 674 |
| polysorbate-80, 0.9% | | | | | | | | |
| sucrose, and 1.8% | | | | | | | | |
| glycine, pH 7.4 | | | | | | | | |
| 10 mM NaPO₄, | 730 | 600 | 791 | 849 | 602 | ND | ND | ND |
| 0.01% polysorbate- | | | | | | | | |
| 80, 1% sucrose, and | | | | | | | | |
| 2% mannitol, pH 7.4 | | | | | | | | |
| 10 mM Tris, 0.01% | 625 | 680 | 660 | 704 | 845 | 849 | 652 | 762 |
| polysorbate-80, 1% | | | | | | | | |
| sucrose, and 2% | | | | | | | | |
| mannitol, pH 7.4 | | | | | | | | |

### C. Long-Term Stability of IL-13R-Ig Formulation

Purified IL-13R was formulated at 10 mg/ml in 1% sucrose, 2% mannitol, 40 mM NaCl, 0.01% polysorbate-80, and 10 mM Tris pH 7.4. 5 ml tubing vials were filled with 1 ml each of the formulation and lyophilized in a Lyo-Star™ development dryer. Lyophilized vials were stored at 2-8° C., 25° C., or 40° C. for up to 24 weeks, with samples analyzed at 0, 4, 7, 12, and 24 weeks. Samples were assayed for appearance (before and after reconstitution), pH, protein concentration (by A280), HMW (by SEC-HPLC), and bioactivity (1050). As shown in Tables 15-17, the IL-13R-Ig formulation is stable under the conditions tested for at least 24 weeks. Table 15 shows results obtained after storage at 2-8° C. Table 16 shows results obtained after storage at 25° C. Table 17 shows results obtained after storage at 40° C.

TABLE 15
Stability of IL-13R-Ig Formulation at 2-8° C.

| | | Appearance | | | | |
|---|---|---|---|---|---|---|
| | Appearance | (after | | Protein | % | |
| | (before reconstitution) | reconstitution) | pH | conc. | HMW | IC50 |
| Specification | White cake, | Solution, | 6.9-7.9 | Report | ≦10% | ≦2200 pM |
| | essentially free of | essentially free of | | results | HMW | |
| | plainly visible | plainly visible | | (target: | | |
| | particulate matter, | particulate matter | | 10 mg/ml) | | |
| | moisture, and | | | | | |
| | container/closure | | | | | |
| | defects | | | | | |
| Initial | Meets specification | Meets specification | 7.37 | 10.58 | 4.76% | 411 pM |
| 4 weeks | Meets specification | Meets specification | 7.36 | 10.33 | 5.34% | 471 pM |
| 7 weeks | Meets specification | Meets specification | 7.38 | 9.48 | 4.18% | 429 pM |
| 12 weeks | Meets specification | Meets specification | 7.38 | 9.96 | 3.19% | 467 pM |
| 24 weeks | Meets specification | Meets specification | 7.42 | 10.0 | 4.03% | 890 +/− |
| | | | | | | 239 pM (10 |
| | | | | | | replicates) |

TABLE 16
Stability of IL-13R-Ig Formulation at 25° C.

| | | Appearance | | | | |
|---|---|---|---|---|---|---|
| | Appearance | (after | | Protein | % | |
| | (before reconstitution) | reconstitution) | pH | conc. | HMW | IC50 |
| Specification | White cake, | Solution, | 6.9-7.9 | Report | ≦10% | ≦2200 pM |
| | essentially free of | essentially free of | | results | HMW | |
| | plainly visible | plainly visible | | (target: | | |
| | particulate matter, | particulate matter | | 10 mg/ml) | | |
| | moisture, and | | | | | |
| | container/closure | | | | | |
| | defects | | | | | |
| Initial | Meets specification | Meets specification | 7.37 | 10.58 | 4.76% | 411 pM |
| 4 weeks | Meets specification | Meets specification | 7.36 | 11.09 | 5.81% | 445 pM |
| 7 weeks | Meets specification | Meets specification | 7.40 | 9.26 | 4.43% | 336 pM |
| 12 weeks | Meets specification | Meets specification | 7.42 | 10.2 | 3.37% | 261 pM |
| 24 weeks | Meets specification | Meets specification | 7.37 | 10.09 | 4.21% | 966 +/− |
| | | | | | | 314 pM (4 |
| | | | | | | replicates |

TABLE 17
Stability of IL-13R-Ig Formulation at 40° C.

| | Appearance | Appearance | | Protein | % | |
|---|---|---|---|---|---|---|
| | (before reconstitution) | (after reconstitution) | pH | conc. | HMW | IC50 |
| Specification | White cake, essentially | Solution, essentially | 6.9-7.9 | Report | ≦10% | ≦2200 pM |
| | free of plainly visible | free of plainly visible | | results | HMW | |
| | particulate matter, | particulate matter | | (target: | | |
| | moisture, and | | | 10 mg/ml) | | |
| | container/closure | | | | | |
| | defects | | | | | |
| Initial | Meets specification | Meets specification | 7.37 | 10.58 | 4.76% | 411 pM |
| 4 weeks | Meets specification | Meets specification | 7.37 | 10.81 | 6.02% | ND |
| 7 weeks | Meets specification | Meets specification | 7.40 | 10.24 | 4.88% | ND |
| 12 weeks | Meets specification | Meets specification | 7.39 | 9.60 | 3.65% | 469 pM |
| 24 weeks | Meets specification | Meets specification | 7.4 | 10.44 | 4.85% | 823 +/− |
| | | | | | | 299 pM (10 |
| | | | | | | replicates) |

The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention. Unless otherwise stated, all percentages refer to weight/weight amounts.

1-45. (canceled)

46. A pharmaceutical composition comprising:
a) an IL-21R-Ig fusion protein,
b) glycine,
c) a disaccharide,
d) a surfactant, and
e) a buffer;
wherein the composition contains 0.5 to 5% glycine, 0.5 to 5% disaccharide, and 0.001 to 0.5% surfactant.

47. The composition of claim 46, further comprising NaCl at a concentration of 1-200 mM NaCl.

48. The composition of claim 46, wherein the concentration of the IL-21R-Ig fusion in the composition is 0.025 to 60 mg/ml.

49. The composition of claim 46, wherein the disaccharide is sucrose or trehalose.

50. The composition of claim 46, wherein the surfactant is polysorbate.

51. The composition of claim 46, wherein the buffer concentration in the composition is 5 to 30 mM.

52. The composition of claim 46, wherein the buffer is a histidine buffer, a tris buffer, or a phosphate buffer.

53. The composition of claim 46, wherein the composition does not contain NaCl.

54. The composition of claim 46, wherein the composition has been lyophilized.

55. The composition of claim 46, wherein the composition is stable at −80° to +40° C. for at least 1 week.

56. A pharmaceutical composition comprising:
a) an IL-21R-Ig fusion protein,
b) mannitol,
c) a disaccharide,
d) a surfactant,
e) a buffer, and
wherein the composition contains 0.5 to 5% mannitol, 0.5 to 5% disaccharide, 0.001 to 0.5% surfactant, and less than 35 mM NaCl.

57. The composition of claim 56, wherein the concentration of the IL-21R-Ig fusion in the composition is 0.025 to 60 mg/ml.

58. The composition of claim 56, wherein the disaccharide is sucrose or trehalose.

59. The composition of claim 56, wherein the surfactant is polysorbate.

60. The composition of claim 56, wherein the buffer concentration in the composition is 5 to 30 mM.

61. The composition of claim 56, wherein the buffer is a histidine buffer, a tris buffer, or a phosphate buffer.

62. The composition of claim 56, wherein the composition does not contain NaCl.

63. The composition of claim 56, wherein the composition has been lyophilized.

64. The composition of claim 56, wherein the composition is stable at −80° to +40° C. for at least 1 week.

65. A method of making the composition of claim 46, comprising:
a) freezing the composition to below −40° C.;
b) raising the temperature of the composition to a temperature chosen from the range from −5° C. to −40° C. for a period of time sufficient to promote crystallization of glycine or mannitol in the composition;
c) lowering the temperature of the composition to below −35° C.;
d) establishing a vacuum; and
e) drying the composition at a temperature chosen from the range from −20° C. to +30° C.
